(19) United States
(12) Patent Application Publication
(10) Pub. No.: US 2017/0247665 A1
(43) Pub. Date: Aug. 31, 2017

(54) A BLUETONGUE VACCINE AND METHODS OF MANUFACTURE THEREOF

(71) Applicant: BIOVET PRIVATE LIMITED, Malur (IN)

(72) Inventors: Tapas BHATTACHARYA, Malur (IN); Shree Naravan SINGH, Malur (IN)

(21) Appl. No.: 15/507,588

(22) PCT Filed: Aug. 21, 2015
(86) PCT No.: PCT/IN2015/050098

§ 371 Date: Feb. 28, 2017

(30) Foreign Application Priority Data
Aug. 29, 2014 (IN) 4235/CHE/2014

(51) Int. Cl. C12N 7/00; A61K 39/15

(52) U.S. Cl. CPC C12N 7/00; A61K 39/15; C12N 2720/12134; A61K 2039/5252

(57) ABSTRACT

Disclosed is a novel vaccine formulation for prevention against Bluetongue virus infection in domestic and wild ruminants, cattle, goats, sheep, pigs, and any other mammals for commercial use. The antigen present in the vaccine is an inactivated antigen comprising 5 different serotypes of Bluetongue virus, thereby making a pentavalent vaccine formulation against Bluetongue virus infections. Also disclosed are method of adaptation of Bluetongue virus in suspension culture upto 2000 liters industrial size bioreactors, and inactivation techniques for preparation of the given vaccine formulation with the presence of suitable adjuvants and preservatives.

### FIELD OF INVENTION

The present invention pertains to the field of viral vaccines. Particularly, the present invention relates to industrial production of Bluetongue virus vaccine compositions and processes or methods of manufacture of vaccine composition against Bluetongue virus infections. The present invention relates to processes for production and purification of virus bulk and inactivation techniques of the said Bluetongue virus bulk thereof.

### BACKGROUND OF THE INVENTION

Bluetongue (BT) is a non-contagious arthropod transmitted viral disease of domestic (sheep & goat) and wild ruminants. BT is caused by a double stranded RNA virus belonging to the genus Orbivirus and family Reoviridae. BT is transmitted by about 20 species of culicoides midges which act as biological vectors of BTV. Immunity against one serotype provides little or no protection against other serotypes. This phenomenon along with vector transmission has complicated the control of the disease. Many animals infected by the disease have their tongue and mucous membrane discolored due to cyanosis and hence the name Bluetongue (BT).

BT has been listed as a List A disease by OIE (Office of International Epizootics or

World Organization for Animal Health). List A diseases are those diseases which can spread rapidly and have a considerable impact on the health of livestock. BT inflicts severe economic losses—direct losses due to high mortality (20-50%) and indirect losses due to high morbidity (up to 100%) and associated costs—veterinary costs, reduced weight gain, milk yield, abortions, infertility, poor wool, carcass quality, restricted international movement of animals, animal products and germplasm. Countries that are free of BT restrict imports of live animals or animal products including semen and embryos from countries that may have BT. Cattle can also carry the virus post-infection and still show no clinical signs. The estimated cost for U.S sheep and cattle producers alone is $125 million per year in lost trade and in testing to certify that animals or animal products for export are free from the Bluetongue virus.

BT was first reported in nineteenth century and is now prevalent in India and other parts of the world. Indian Council of Agriculture Research (ICAR) had initiated the All India Network on Bluetongue in 2001 to control this disease. India is home to 21 crore sheep and goat and these animals are an important source of meat, wool, milk, hide etc. Sheep and goat farming is mainly done in rural areas by landless or small and marginal farmers and any impact on their incomes can have disastrous consequences Like other arthropod-borne viruses, there is no “cure” or effective treatment for Bluetongue disease other then treating the clinical signs in an attempt to reduce the severity. Vaccination is the most efficient mitigation measure possible for prevention and eradication of BT. Though BT vaccine is available globally, developing effective and affordable vaccine will help in increasing incomes of millions of sheep and goat farmers across India and other developing countries who rely on agriculture and animal husbandry. A Bluetongue pentavalent vaccine incorporating five prevalent serotypes—BTV1, BTV2, BTV10, BTV16 and BTV23 as a strategy to control the disease will serve as an effective tool to eradicate this desease, and enable prevention from BT from the most prevalent serotypes of this virus.

Live attenuated vaccines are inexpensive to produce in large quantities, generate protective immunity after a single inoculation and have proven effective in preventing clinical BT disease. Adverse consequences are depressed milk production in lactating sheep, and abortion/embryonic death and teratogenesis in offspring from pregnant females that are vaccinated during the first half of gestation. Another risk associated with the use of live attenuated vaccines is their potential for spread by vectors, with eventual reversion to virulence or reassortment of vaccine virus genes with those of wild-type virus strains. The frequency and significance of these events remain poorly defined, but transmission of vaccine strains by vector Culicoides in the field has already been documented in Europe.

Industrial Production of Bluetongue virus vaccine, is also another difficulty. Bluetongue virus production has been earlier done in roller bottles culture. Roller Bottle conventional production methods involves facility complexity, constant cleaning process and the risk of cross-contamination. Possibility of the rapid expansion of the capacity of the production is not feasible. Selection of appropriate culture conditions can be important to achieve consistent cell culture and virus production across sites and scales. The capacity of production in roller bottles is limited, and the available surface area for growth of substrate cells are also very low. For this reason, industrial production of Bluetongue virus has not been possible in developing countries till date including India. Importation costs to check Bluetongue infections are overwhelming. Moreover, production of Bluetongue vaccine in roller bottles also involve high manufacturing cots, owing to operational difficulties and associated human labour. Culture of Bluetongue virus in single use bioreactors have been reported earlier. Although characteristics like tank geometry and hardware are not subject to change during scale-up, the scalability from 50L to 1000L bioreactor can be one of the strategies for production process in single use bioreactors, but single use bioreactors adds to the manufacturing cost of the vaccine although it enables continuous process. Hence it is desirable that, alternative methods of industrial production of Bluetongue vaccine, that reduces the cost of the vaccine and is operationally feasible, with lower culture handling and to achieve more homogeneity. This invention overcomes the difficulties associated with industrial production of Bluetongue vaccine in roller bottle cultures. This invention presents a novel method of virus culture for industrial production of Bluetongue vaccine with BHK-21 in suspension cell lines in continuous culture in stainless steel vessels. BioBT-Oil™ is an inactivated Bluetongue virus antigen adjuvanted with oil providing stable and long-lasting immunity with high protective index. Bluetongue virus serotypes BTV 1, BTV 2, BTV 10, BTV 16 and BTV 23 are used as the pentavalent antigen along with an adjuvant for production of Bluetongue vaccine.

### OBJECTS OF THE INVENTION

One object of the present invention is to provide a novel method to produce stable inactivated vaccine composition that is capable to prevent as well as provide protection from bluetongue virus infections caused by multiple serotypes BTV 1, BTV 2, BTV 10, BTV 16 and BTV 23 of the Bluetongue virus.

Another object of the invention is to provide for a method of adaptation of the Bluetongue virus in Baby Hamster Kidney-21 cell line (BHK-21) using a suspension culture, for industrial production of the Bluetongue vaccine.

Yet another object of the invention is to provide for a suitable method of commercial production of Bluetongue vaccine at specified experimental parameters, and techniques without involving roller bottles at all.

Yet another object of the invention is to provide a method to manufacture a cost effective, Bluetongue virus vaccine to eradicate and to prevent bluetongue virus infection.

### SUMMARY OF THE INVENTION

According to one embodiment of the invention, various methods of culture of BHK-21 cells in suspension cell line is disclosed. Satisfactory levels of growth of BHK-21 at various experimental parameters involving proper media composition and requirement of change in media is optimized, to obtain satisfactory levels of cell count within a desired amount of time in industrial size bioreactors. BHK-21 cells are infected with the Bluetongue virus, and accordingly the embodiment describes, a method of adaptation of bluetongue virus to grow in BHK-21 cells in suspension cell lines.

According to another embodiment of the invention, the adaptation of Bluetongue virus culture from roller bottles to suspension cell lines in SS vessels is scaled up from small-scale culture flasks to next subsequent sizes upto 2000 liter batch size bioreactor to generate production batch size yield of purified Bluetongue virus bulk.

### DETAILED DESCRIPTION OF THE INVENTION

### EXAMPLE 1

### Preparation of Master and Working Seed Virus in BHK21 Suspension Cell Lines

The Bluetongue virus serotypes BTV1, BTV2, BTV10, BTV16 and BTV23 were received from Tamil Nadu Veterinary and Animal Sciences University (TANUVAS), Chennai, India under a Memorandum of Understanding between Indian Council of Agricultural Research and Biovet Private Limited, Bangalore dated 15^{th} February 2011. These strains are the most current and infective strains present in India and are used to produce bulk antigen. The source material from TANUVAS is aliquoted and stored in liquid nitrogen. These virus are sequenced, characterized and certified by TANUVAS. The master and working seed virus is prepared from source material in BHK21 suspension cell lines and stored at liquid nitrogen above vapour phase.

Master Bank

| | | Total | No. of | |
|---|---|---|---|---|
| | Virus | No. of | vials | |
| Sl. No. | Strain | vials | used | Purpose |
| 1 | BTV 1 | 30 | 7 | Identification, Sterility, Virus titer, |
| | | | | Mycoplasma and Working Bank |
| 2 | BTV 2 | 30 | 7 | Identification, Sterility, Virus titer, |
| | | | | Mycoplasma and Working Bank |
| 3 | BTV 10 | 30 | 7 | Identification, Sterility, Virus titer, |
| | | | | Mycoplasma and Working Bank |
| 4 | BTV 16 | 30 | 7 | Identification, Sterility, Virus titer, |
| | | | | Mycoplasma and Working Bank |
| 5 | BTV 23 | 30 | 7 | Identification, Sterility, Virus titer, |
| | | | | Mycoplasma and Working Bank |

Working Bank

| | | Total | No. of | |
|---|---|---|---|---|
| | Virus | No. of | vials | |
| Sl. No. | Strain | vials | used | Purpose |
| 1 | BTV 1 | 80 | 9 | Identification, Sterility, Virus titer, |
| | | | | Mycoplasma and Antigen batch |
| | | | | preparation |
| 2 | BTV 2 | 80 | 9 | Identification, Sterility, Virus titer, |
| | | | | Mycoplasma and Antigen batch |
| | | | | preparation |
| 3 | BTV 10 | 80 | 9 | Identification, Sterility, Virus titer, |
| | | | | Mycoplasma and Antigen batch |
| | | | | preparation |
| 4 | BTV 16 | 80 | 9 | Identification, Sterility, Virus titer, |
| | | | | Mycoplasma and Antigen batch |
| | | | | preparation |
| 5 | BTV 23 | 80 | 9 | Identification, Sterility, Virus titer, |
| | | | | Mycoplasma and Antigen batch |
| | | | | preparation |

### Characterization of Master and Working Seed Bank:

The following tests were carried out for the master and working seed virus, identification of all Bluetongue virus serotypes has been done. Sterility test is done as per I.P.2010 to check the presence of bacteria and fungi by Fluid Thioglycollate Medium and Soybean Casein Digest Medium at 35° C. and 25° C. respectively.

| | | | Working | |
|---|---|---|---|---|
| Sl. No. | Master Bank | Result | Bank | Result |
| 1 | BTV 1 | Sterile | BTV 1 | Sterile |
| 2 | BTV 2 | Sterile | BTV 2 | Sterile |
| 3 | BTV 10 | Sterile | BTV 10 | Sterile |
| 4 | BTV 16 | Sterile | BTV 16 | Sterile |
| 5 | BTV 23 | Sterile | BTV 23 | Sterile |

Virus titer is checked for all the Bluetongue virus serotypes by TCID50 method as per in-house specification in Biovet QC lab.

| | | | Working | |
|---|---|---|---|---|
| Sl. No. | Master Bank | Result | Bank | Result |
| 1 | BTV 1 | 10^{-5.47}TCID₅₀/ml | BTV 1 | 10^{-5.77}TCID₅₀/ml |
| 2 | BTV 2 | 10^{-5.87}TCID₅₀/ml | BTV 2 | 10^{-6.12}TCID₅₀/ml |
| 3 | BTV 10 | 10^{-6.12}TCID₅₀/ml | BTV 10 | 10^{-6.20}TCID₅₀/ml |
| 4 | BTV 16 | 10^{-6.30}TCID₅₀/ml | BTV 16 | 10^{-6.30}TCID₅₀/ml |
| 5 | BTV 23 | 10^{-5.87}TCID₅₀/ml | BTV 23 | 10^{-6.12}TCID₅₀/ml |

The mycoplasma test is done and absence of mycoplasma was ascertained.

### EXAMPLE 2

### Large Scale Production of BHK21 Suspension Cell Lines and Scale Up for Bluetongue Vaccine

One vial of BHK-21 C13 cells from the working cell bank is taken and is thawed in a water bath at 36° C.±1° C. for 3 to 5 minutes and serially scaled up in cell growth medium. The cell growth medium comprises of Glassgow Essential Medium and new born calf serum or adult bovine serum. Initially, T-75, T-175 tissue culture flasks are used followed by 500 ml, 1 L, 5 L, 10 L, 15 L glass bottles and later in large scale production level batch sizes of 200 L and 2000 L. BHK-21 suspension cells are aseptically transferred into Virus Bioreactor. The cells are then subjected to chilling and sedimentation for a period of 18-24 hours and later the spent media is removed from cell suspension.

### Bluetongue Virus:

Bluetongue virus seed of intended serotype from working seed bank is thawed with a titer value not less than 10^{-5.0} Tissue culture infectious dose (TCID₅₀) is infected as per Multiplicity of Infection (MOI) calculation and followed by virus maintenance medium without any serum. Thereafter, Bluetongue virus is inoculated into BHK-21 C13 suspension cells and kept for incubation at 36° C. for 48-72 hours with continuous stirring along with cell control. Virus culture is harvested when it attains 90-95% Cytopathic Effect (CPE). The viral harvest from bioreactor is subjected to centrifugation to remove cell debris. After complete infection the virus culture is centrifuged, the supernatant collected into a separate sterile container and used as seed for the large scale production.

The standard virus titer results in the Suspension cell lines of the Bluetongue virus are mentioned in the below table:

| Sl. No. | Serotypes | Virus titer result | Sterility |
|---|---|---|---|
| 1 | BTV 1 | 10^{-5.12}TCID₅₀/ml | Sterile |
| 2 | BTV 2 | 10^{-5.87}TCID₅₀/ml | Sterile |
| 3 | BTV 10 | 10^{-5.42}TCID₅₀/ml | Sterile |
| 4 | BTV 16 | 10^{-6.12}TCID₅₀/ml | Sterile |
| 5 | BTV 23 | 10^{-4.87}TCID₅₀/ml | Sterile |

Scale up experimental data from T-175cm² tissue culture flask to large scale production of Bluetongue virus equivalent to 2000 liter batch-size of five Indian isolates in BHK-21 suspension cell lines in bioreactor is presented in the below mentioned table:

TABLE 1
Culture of Bluetongue virus in suspension cell lines and scale up from TC flask to
2000 liter commercial scale batch size.

| | | 15 liter batch | 250 L | 250 L | 2000 L | 2000 L |
|---|---|---|---|---|---|---|
| | | in | batch-I in | batch-II in | batch-I in | batch-II in |
| | | Suspension | Suspension | Suspension | Suspension | Suspension |
| | T-175 cm² | Cells | Cells | Cells | Cells | Cells |
| | TC Flask | (BHK-21) | (BHK-21) | (BHK-21) | (BHK-21) | (BHK-21) |
| | Media conc. | | | | | |
| | GMEM for | GMEM for | GMEM for | GMEM for | GMEM for | GMEM for |
| | cell growth | cell growth | cell growth | cell growth | cell growth | cell growth |
| | with 8% v/v | with 8% v/v | with 8% v/v | with 8% v/v | with 8% v/v | with 8% v/v |
| Time | NBCS | ABS | ABS | ABS | ABS | ABS |
| Cell count at | 0.4 × 10⁵ cells/ml | 0.65 × 10⁶ cells/ml | 0.55 × 10⁶ cells/ml | 0.6 × 10⁶ cells/ml | 0.5 × 10⁶ cells/ml | 0.6 × 10⁶ cells/ml |
| 0 Hour | | | | | | |
| Cell count at | 1.3 × 10⁶ cells/ml | 1.50 × 10⁶ cells/ml | 1.4 × 10⁶ cells/ml | 1.5 × 10⁶ cells/ml | 1.5 × 10⁶ cells/ml | 1.6 × 10⁶ cells/ml |
| 24 Hour | | | | | | |
| Dicanting of | Addition of | Addition of | Addition of | Addition of | Addition of | Addition of |
| spent media | GMEM | GMEM | GMEM | GMEM | GMEM | GMEM |
| and seed | without serum | without serum | without serum | without serum | without serum | without serum |
| Virus | for virus | for virus | for virus | for virus | for virus | for virus |
| innoculation | multiplication | multiplication | multiplication | multiplication | multiplication | multiplication |
| 48 Hours | 10^{-4.90}TCID₅₀ | 10^{-4.87}TCID₅₀ | 10^{-5.24}TCID₅₀ | 10^{-5.12}TCID₅₀ | 10^{-4.50}TCID₅₀ | 10^{-4.87}TCID₅₀ |
| after | per ml of | per ml of | per ml of | per ml of | per ml of | per ml of |
| infection | BTV-1 | BTV-1 | BTV-1 | BTV-1 | BTV-1 | BTV-1 |
| | 10^{-5.24}TCID₅₀ | 10^{-5.42}TCID₅₀ | 10^{-5.87}TCID₅₀ | 10^{-5.47}TCID₅₀ | 10^{-5.12}TCID₅₀ | 10^{-5.29}TCID₅₀ |
| | per ml of | per ml of | per ml of | per ml of | per ml of | per ml of |
| | BTV-2 | BTV-2 | BTV-2 | BTV-2 | BTV-2 | BTV-2 |
| | 10^{-4.80}TCID₅₀ | 10^{-5.80}TCID₅₀ | 10^{-5.47}TCID₅₀ | 10^{-5.40}TCID₅₀ | 10^{-5.15}TCID₅₀ | 10^{-5.31}TCID₅₀ |
| | per ml of | per ml of | per ml of | per ml of | per ml of | per ml of |
| | BTV-10 | BTV-10 | BTV-10 | BTV-10 | BTV-10 | BTV-10 |
| | 10^{-5.80}TCID₅₀ | 10^{-6.12}TCID₅₀ | 10^{-5.88}TCID₅₀ | 10^{-5.80}TCID₅₀ | 10^{-5.87}TCID₅₀ | 10^{-5.50}TCID₅₀ |
| | per ml of | per ml of | per ml of | per ml of | per ml of | per ml of |
| | BTV-16 | BTV-16 | BTV-16 | BTV-16 | BTV-16 | BTV-16 |
| | 10^{-5.87}TCID₅₀ | 10^{-6.12}TCID₅₀ | 10^{-5.87}TCID₅₀ | 10^{-5.40}TCID₅₀ | 10^{-5.47}TCID₅₀ | 10^{-5.86}TCID₅₀ |
| | per ml of | per ml of | per ml of | per ml of | per ml of | per ml of |
| | BTV-23 | BTV-23 | BTV-23 | BTV-23 | BTV-23 | BTV-23 |
| 72 Hours | 10^{-5.24}TCID₅₀ | 10^{-5.47}TCID₅₀ | 10^{-5.47}TCID₅₀ | 10^{-5.48}TCID₅₀ | 10^{-5.50}TCID₅₀ | 10^{-5.85}TCID₅₀ |
| after | per ml of | per ml of | per ml of | per ml of | per ml of | per ml of |
| infection | BTV-1 | BTV-1 | BTV-1 | BTV-1 | BTV-1 | BTV-1 |
| | 10^{-5.87}TCID₅₀ | 10^{-5.87}TCID₅₀ | 10^{-6.12}TCID₅₀ | 10^{-6.00}TCID₅₀ | 10^{-5.47}TCID₅₀ | 10^{-5.86}TCID₅₀ |
| | per ml of | per ml of | per ml of | per ml of | per ml of | per ml of |
| | BTV-2 | BTV-2 | BTV-2 | BTV-2 | BTV-2 | BTV-2 |
| | 10^{-5.12}TCID₅₀ | 10^{-6.12}TCID₅₀ | 10^{-6.12}TCID₅₀ | 10^{-6.20}TCID₅₀ | 10^{-6.30}TCID₅₀ | 10^{-6.32}TCID₅₀ |
| | per ml of | per ml of | per ml of | per ml of | per ml of | per ml of |
| | BTV-10 | BTV-10 | BTV-10 | BTV-10 | BTV-10 | BTV-10 |
| | 10^{-6.12}TCID₅₀ | 10^{-6.30}TCID₅₀ | 10^{-6.30}TCID₅₀ | 10^{-6.12}TCID₅₀ | 10^{-6.39}TCID₅₀ | 10^{-6.30}TCID₅₀ |
| | per ml of | per ml of | per ml of | per ml of | per ml of | per ml of |
| | BTV-16 | BTV-16 | BTV-16 | BTV-16 | BTV-16 | BTV-16 |
| | 10^{-6.00}TCID₅₀ | 10^{-6.39}TCID₅₀ | 10^{-6.39}TCID₅₀ | 10^{-6.12}TCID₅₀ | 10^{-6.12}TCID₅₀ | 10^{-6.30}TCID₅₀ |
| | per ml of | per ml of | per ml of | per ml of | per ml of | per ml of |
| | BTV-23 | BTV-23 | BTV-23 | BTV-23 | BTV-23 | BTV-23 |

### EXAMPLE 3

### Inactivation of Bluetongue Virus

The clarified virus from 2000 Liter batch size is inactivated with 0.04% v/v formalin for 1 hour followed by addition of 1.5 mM Binary Ethyleneimine (BEI) to the final culture volume at 0 hour and kept under continuous stirring for 24 hours at 36±1 ° C. The culture was transferred to another sterile vessel and second dose of BEI is added having a final concentration of 3 mM and kept for a period of another 24 hours under stirring at 36±1 ° C. The samples were collected at different intervals of time (0, 1^{st}, 2^{nd}, 3^{rd}, 4^{th}, 5^{th}, 6^{th}, 24^{th} , and 48^{th} hours) for studying RNA degradation by using 1% Agarose gel electrophoresis method. FIGS. 1 and 2 shows that there is no presence of RNA after 24 hours of inactivation and 48 hours of post inactivation on respective lanes.

Concentration of Bluetongue virus: After inactivation, the virus brew is subjected to final concentration of approx 30× using Tangential Flow Filtration (TFF) system and the concentrated bluetongue antigen is collected in a 100 L pressure vessel. The collected antigen in 100 liter pressure vessel is further dispensed under Biosafety cabinet into sterile Polypropylene (PP) bottles and stored at 2° C.-8° C. for vaccine blending. Samples are collected for quality control testing on various parameters like sterility and Indirect ELISA and it was found to be stable for period of 2 years at 5±3° C.

We claim:

1. A method of industrial cultivation of Bluetongue virus, the said method comprising, virus culture of Bluetongue virus in suspension BHK-21 cell lines in stainless steel vessels industrial bioreactor.

2. A method of adaptation of Bluetongue virus in BHK-21 cells in suspension culture capable of industrial production of Bluetongue vaccine.

3. A method of scale up of virus culture of Bluetongue virus on BHK-21 cells from T-175 flask to an industrial bioreactor of 250 liter batch volume, wherein the virus titer attained of the Bluetongue virus ranges from 10^{-5.5}TCID₅₀/ml to 10^{-7.0}TCID₅₀/ml within 48 hours to 72 hours of infection.

4. A method of scale up of virus culture of Bluetongue virus on BHK-21 cells from 250 liter batch volume to an industrial bioreactor of 2000 liter batch volume, wherein the virus titer attained of the Bluetongue virus ranges from 10^{-5.5}TCID₅₀/ml to 10^{-7.0}TCID₅₀/ml within 48 hours to 72 hours of infection.

5. A vaccine composition for the prophylaxis of animals from Bluetongue virus infection, comprising a vaccine antigen, wherein the said vaccine antigen comprises of Bluetongue virus serotypes BTV 1, BTV 2, BTV 10, BTV 16 and BTV 23, the said Bluetongue serotypes being adapted to infect BHK-21 cells in suspension cell line culture to obtain a virus titer from 10^{-5.5}TCID₅₀/ml to 10^{-7.0}TCiD₅₀/ml within 48 hours to 72 hours of infection thereby capable of conferring immune response against Bluetongue virus infections.

6. The vaccine composition according to claim 1, wherein the animal is a domestic or a wild ruminant, cattle, goat, sheep, pig, or any other mammal involved in animal husbandry useful for commercial benefit.

7. The vaccine formulation according to claim 5, wherein the vaccine antigen is inactivated using Binary Ethylenimine.

8. The vaccine formulation according to claim 5, wherein the vaccine composition further comprises a vaccine adjuvant.

9. The vaccine composition according to claim 5, is stable for period of 1 year at 5±3° C.
